(19) United States
(12) Patent Application Publication
(10) Pub. No.: US 2025/0279192 A1
(43) Pub. Date: Sep. 4, 2025

(54) TRANSITION OF CARE WORK FLOW AND PRIORITIZATION SYSTEM

(71) Applicant: CITYBLOCK HEALTH, INC., Brooklyn, NY (US)

(72) Inventors: Sheila Turbides MINAYA, Richmond, VA (US); Dhakshila PARAMANATHAN, Brooklyn, NY (US); Myung WOO, Durham, NC (US); Eric HILTON, Whitefish, MT (US); Aarthi RAO, Concord, MA (US); Elizabeth HOFFMAN, Brooklyn, NY (US)

(21) Appl. No.: 19/067,461

(22) Filed: Feb. 28, 2025

Related U.S. Application Data
(60) Provisional application No. 63/560,462, filed on Mar. 1, 2024.

(51) Int. Cl. G16H 40/20; G16H 15/00

(52) U.S. Cl. CPC G16H 40/20; G16H 15/00

(57) ABSTRACT

An example method described herein includes receiving discharge data for a patient from a healthcare facility discharging the patient and predicting a likelihood of readmission for the patient based on the discharge data using a care transition model, where the care transition model is trained using historical discharge data and historical readmission data associated with a plurality of patients and a plurality of healthcare facilities. The method further includes determining a priority for the patient based on the likelihood of readmission. A care services system is also disclosed which includes a systems interface configured to receive discharge data for a patient from a healthcare facility system discharging the patient and a care transition model configured to predict a likelihood of readmission for the patient based on the discharge data, and a prioritization element configured to determine a priority of the patient based on the likelihood of readmission.

### CROSS-REFERENCE TO RELATED APPLICATIONS

This application claims the benefit of priority pursuant to 35 U.S.C. § 119(e) to U.S. Provisional Patent Application No. 63/560,462, filed Mar. 1, 2024, entitled “Transition of Care Work Flow and Prioritization System,” which is hereby incorporated by reference herein in its entirety. This application is related to U.S. application Ser. No. 19/067,272, filed on Feb. 28, 2025, entitled “Identification of Health Risk and Impact Assessment,” which is hereby incorporated by reference herein in its entirety.

### BACKGROUND

Many people are discharged from healthcare facilities every day and due to a variety of factors, many of the same people are readmitted to healthcare facilities for reasons that may be preventable. The healthcare facility systems maintaining the discharge data, admission data, and electronic health records (EHR) may not be configured for intercommunication, such that the information may be aggregated and tracked. Care teams may not have all of the information pertaining to people at risk for readmission to healthcare facilities.

Additionally, patients, especially those in certain vulnerable populations, may be wary of unknown people reaching out to contact them, e.g., to schedule appointments, check in after procedures, or the like. Such wariness can mean that many reach out attempts are not successful, which can be detrimental to the patient's overall health, health care experiences, or recovery.

### SUMMARY

In one embodiment, a computer implemented method is disclosed. The method includes training a care transition model using historical discharge data and historical readmission data associated with a plurality of patients and a plurality of healthcare facilities; receiving by a processor discharge data for a patient from a healthcare facility discharging the patient after a health event; predicting by the processor a likelihood of readmission for the patient to a healthcare facility based on the discharge data using the care transition model; and outputting a readmission profile for the patient to a user display, where the readmission profile for the patient is based on the likelihood of readmission for the patient.

In some embodiments, the method may include receiving by the processor admission data for the patient when the patient is admitted to the healthcare facility. The method may predict the likelihood of readmission for the patient to the healthcare facility using the care transition model and output the readmission profile for the patient, where, when the patient is discharged from the healthcare facility, the likelihood of readmission for the patient and the readmission profile of the patient are updated with information related to the discharge.

In another example, one or more transitory computer readable media encoded with instructions, which when executed by one or more processors of a care services system cause the care services system to receive discharge data for a patient from a healthcare facility discharging the patient, predict a likelihood of readmission for the patient based on the discharge data using a care transition model, where the care transition model is trained using historical discharge data and historical readmission data associated with a plurality of patients and a plurality of healthcare facilities, and determine a priority for the patient based on the likelihood of readmission.

In some embodiments, the one or more transitory computer readable media may receive admission data for the patient when the patient is admitted to the healthcare facility. The method may predict the likelihood of readmission for the patient to the healthcare facility using the care transition model and determine a priority for the patient, where, when the patient is discharged from the healthcare facility, the likelihood of readmission for the patient and the priority of the patient are updated with information related to the discharge.

In another embodiment, a computer implemented method is disclosed. The method includes receiving by a processor a request to generate a confidence metric associated with a patient discharged from a healthcare facility after a health event, retrieving by the processor contact information associated with the patient from a database, analyzing by the processor a history of contact associated with the patient and the contact information associated with the patient, and generating a recommendation as to which contact information to use to contact the patient. For example, the recommendation may be output to a user device as a most likely to reach patient suggestion or notification.

In another embodiment, a computer implemented method is disclosed. The method may include receiving by a processor contact information associated with a patient and a target data of contact, determining by the processor a reason for the contact, determining by the processor a pre-contact notification frequency based on the reason for the contact and the contact information associated with the patient, generating by the processor a pre-contact notification schedule, and transmitting the pre-contact notifications to the patient ahead of the target date of the contact according to the pre-contact notification schedule.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments and implementations and illustrated in the accompanying drawings.

### BRIEF DESCRIPTION OF DRAWINGS

FIG. 1 illustrates an example system including a care services system in accordance with various embodiments of the disclosure.

FIG. 2 illustrates an example care services system in communication with various other systems.

FIG. 3 illustrates an example computing system that may be used for implementing various embodiments of the disclosure.

FIG. 4 illustrates an example process for generation of a likelihood of readmission and a priority for a discharged patient by a care services system.

FIG. 5 illustrates an example process for generating a confidence metric for a patient discharged from a healthcare facility.

FIG. 6 illustrates an example process for transmitting pre-contact notifications to a patient ahead of a scheduled contact date after the patient is discharged from a healthcare facility.

FIG. 7 depicts an example pre-contact notification as displayed on a user device according to some embodiments of the disclosure.

### DETAILED DESCRIPTION

Care teams (e.g., for health care services, such as doctors, nurses, and other supporting people) may follow up with people (e.g., call, visit, or the like) who have been discharged from a healthcare facility in order to prevent the need for that person to be readmitted to the same healthcare facility (e.g., hospital, surgery center, emergency room, etc.) or another for the same or another health issue. Repeated readmission to healthcare can be expensive, not only in terms of money but in time and healthcare personnel, as well as impact certain metrics healthcare providers may be measured against.

In some instances, healthcare facility discharge data, admission data, and electronic health records (EHR) for patients is not readily or easily shared with care teams or with other healthcare facilities, e.g., laws or regulations may prevent or limit data exchange. The point at which a person transitions through the healthcare system, such as when a patient is discharged from a facility, is a critical point for people with a higher likelihood of readmission. If care teams can identify people with a higher risk of readmission at the point of transition, the care team may be able to intervene and prevent a readmission. It should be noted that the care team generally may not include the hospital care team, e.g., may include a general practitioner, nurse, or other provider that is associated with the patient's care, but not necessarily providing the care within the hospital or other institution providing emergency or specialized care.

Further, readmission risk is not assessed or is done on an ad hoc basis based on a particular provider recommending an individual course of action for a particular patient. The present disclosure includes system and methods to automatically identify and make recommendations for high readmission risk patients. For example, by aggregating discharge data, admission data, EHR, and other information about people transitioning through healthcare facilities, a machine learning model can be trained to make predictions regarding a discharge event (e.g., with respect to a discharge of a patient, can assess the risk of readmission to a healthcare facility with respect to a health event for which that person was discharged). Based on the prediction, alerts, notifications, and/or triggers can be implemented within a health care platform to transmit information between providers and ensure that a high-risk patient is transitioned appropriately. It should be noted that in some implementations, the model may be trained to assess a readmission risk based on a particular discharge event, which may be based in part on the patient's characteristics and other features of the admission leading to the discharge.

In some embodiments, the model may be trained to assess the readmission risk based on a particular admission event, such as the likelihood of readmission following the upcoming discharge associated with the admission. The model may impute information related to the admission to assess the readmission risk. In other embodiments, the model may be trained to predict a particular patient's risk for being readmitted.

For example, care teams, which are often understaffed, may use the system to prioritize the people who require follow up after being discharged from a healthcare facility, e.g., effectively allocate care resources. Following up may include scheduling a meeting, telephone calls, text messages, letters, emails, and the like from the care team to assist the person with situations that may arise that compel them to return to a healthcare facility.

Additionally, there are patient populations that may be underserved from a healthcare perspective, including people in low or poor economic conditions, people of color, and the like. Such populations may be hesitant to engage with certain health providers based on prior experience, lack of access, etc. Increasing engagement with such populations, as well as increasing people's access to overall health services can be immensely helpful. However, determining contact information, especially for people that may move frequently, that do not have ready access to a computer or smart phone, and/or change contact numbers frequently may be difficult.

Various embodiments include systems to address both issues. For example, in one embodiment, the system can make predictions to determine patients that are more likely to respond to an outreach attempt, such as those most likely to answer a phone call. The predictions may be based on patient contact history, patient provided information, and the like. This helps to reduce hours required by outreach teams and helps to ensure patients are contacted timely, which is important for many health conditions. For example, in certain instances the system may be configured to rank a particular outreach patient higher if the contact information for that patient is predicted to be one that is likely to be answered (e.g., valid). The system rank may also take into account prioritization based on readmission risk and/or other factors.

As another example, patients may be “primed” for engagement with certain providers. For instance, the system may provide advance notifications to such patients that include details regarding future contact to help increase the engagement percentage for patient outreach. Such priming further helps to ensure high engagement with patients, as patients may be more likely to answer a phone call or notification after receiving one or more advanced notifications indicating the timing and reason for the outreach. The system may be configured to time and prioritize the priming notifications based on patient characteristics, prioritization, and the like.

In examples where the admission of the patient to the healthcare facility is used to predict the likelihood of readmission, healthcare providers may engage with the patient prior to discharge from the healthcare facility based on the patient's risk of readmission and/or priority. For example, if the likelihood of readmission for the patient is high, such as above a threshold level, when the patient is admitted to the healthcare facility, the care team may engage with the patient before the upcoming discharge of the patient from the healthcare facility and while the location of the patient is known to the care team.

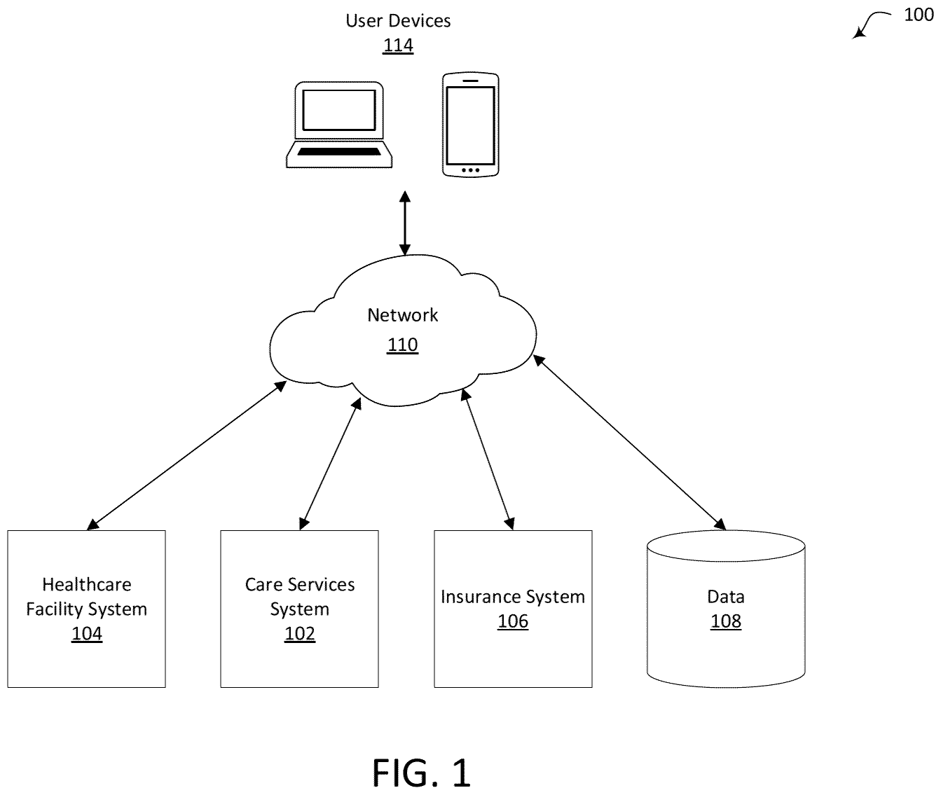

Turning now to the drawings, FIG. 1 illustrates an example system 100 including a care services system 102 in accordance with various embodiments of the disclosure. The care services system 102 may generally communicate with various systems, such as healthcare facility systems 104, insurance systems 106, and various data sources, such as datastore 108, to generate a readmission profile for a patient after a health event. Health events may be, for example, illnesses, traumatic injuries, rehabilitation, mental health issues, pregnancies, strokes, heart attacks, and the like. The readmission profile may include information about the patient's health events, contact information associated with the patient, a care transition plan, alerts, notifications, data tags, and/or triggers. The readmission profile may be transmitted to a user device 114, such as a care team device, to be used by care team members and/or others to implement care services.

In various examples, each of the healthcare facility systems 104, the insurance systems 106, and the user devices 114 may communicate with the care services system 102 via the network 110. The care services system 102 may generally serve as an interface between the healthcare facility systems 104 (e.g., hospital or provider systems), the insurance systems 106, and the user device 114. For example, the care services system 102 may receive historical discharge and historical readmission data associated with a plurality of patients from a plurality of healthcare facility systems 104 and/or insurance data from a plurality of insurance systems 106. The care services system 102 may use the historical discharge data and historical readmission data to train a care transition model used to generate readmission profiles for particular patients. The readmission profiles may, in various examples, be output to user devices 114. For example, the care services system may output a list of patients for follow up to the user device 114 based on the readmission profiles associated with the patients. A user, such as a care team member, may then use the list to create and execute a plan for contacting the patients.

The care services system 102 may be generally implemented by a computing device or combinations of computing resources in various embodiments. In various examples, the care services system 102 may be implemented by one or more servers, cloud computing resources, and/or other computing devices. The care services system 102 may, for example, use various processing resources to communicate with various services, access data, configure user portals, create and/or use building digital assets, and the like. The care services system 102 may further include memory and/or storage locations to store program instructions for execution by the processor and various data used by the care transition model.

The healthcare facility system 104 and the insurance system 106 may similarly be implemented by one or more computing devices or combinations of computing resources in various embodiments. Healthcare facility systems 104 may include, in various examples, systems used by hospitals, clinics, doctors' offices, rehabilitation facilities, urgent care facilities, and the like. These systems may be patient intake software, software used to create electronic health records (EHR), an admission-discharge-transfer (ADT) system, and the like. In some examples, the systems may further label ADT transmissions as inpatient events. In some examples, a healthcare facility, such as a hospital, may use multiple systems to maintain patient information. The insurance system 106 may include, in various examples, systems used by health insurance companies to maintain client information. For example, a health insurance company may use software to maintain records pertaining to client coverage and claims.

The network 110 may be implemented using one or more of various systems and protocols for communications between computing devices. In various embodiments, the network 110 or various portions of the network 110 may be implemented using the Internet, a local area network (LAN), a wide area network (WAN), cloud networking, and/or other networks. In addition to traditional data networking protocols, in some embodiments, data may be communicated according to protocols and/or standards including near field communication (NFC), Bluetooth, cellular connections, and the like.

Generally, the user devices 114 may be devices belonging to end users. For example, the user device 114 may be used by a care team member and/or patient or member being contacted to receive health services. In various implementations, the user device 114 may be implemented using any number of computing devices including, but not limited to, a computer, a laptop, tablet, mobile phone, smart phone, wearable device (e.g., AR/VR headset, smart watch, smart glasses, or the like), smart speaker, vehicle (e.g., automobile), or appliance. Generally, the user devices 114 may include one or more processors, such as a central processing unit (CPU) and/or graphics processing unit (GPU). The user devices 114 may generally perform operations by executing executable instructions (e.g., software) using the processor(s).

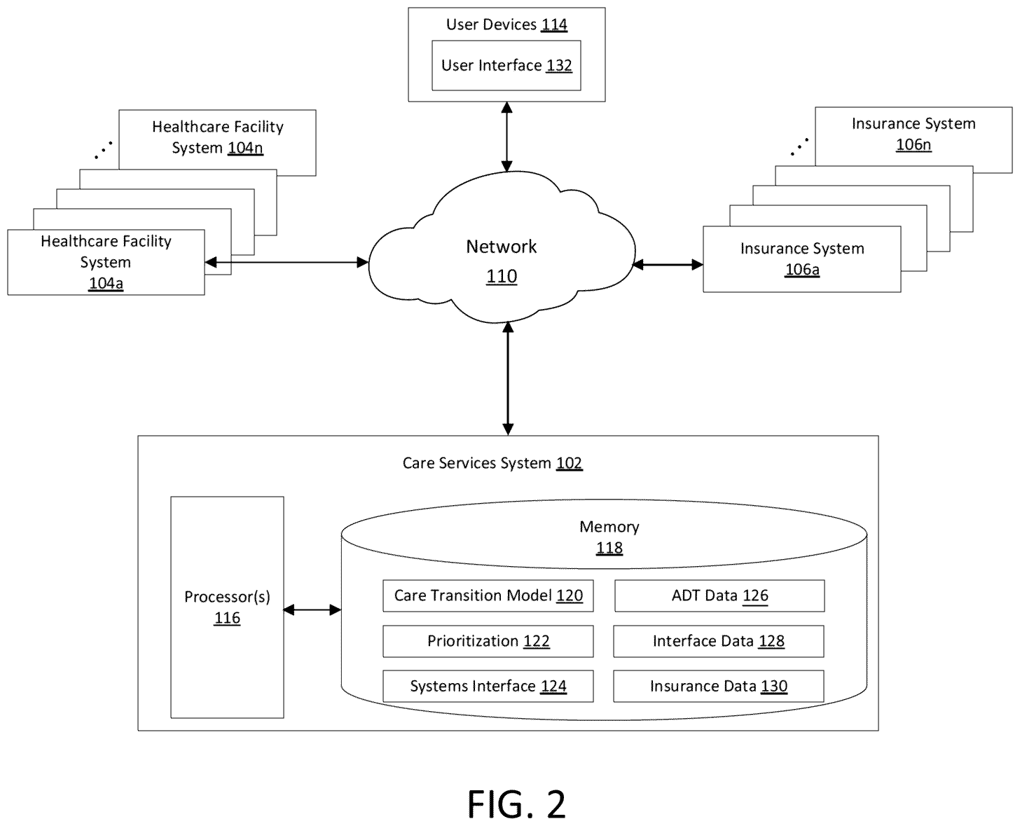

FIG. 2 illustrates an example care services system 102 in communication with various other systems. The care services system 102 generally includes various modules and/or components for performing various functions of the care services system 102. For example, the care services system 102 may include a systems interface 124 for communication with various healthcare facility systems 104a-104n and/or various insurance systems 106a-106n. For example, the systems interface 124 may receive historical discharge and readmission data associated with a patient and/or a plurality of patients and/or current discharge data and/or admission data associated with a patient from one or more of the healthcare facility systems 104a-104n. The care services system 102 may further include a care transition model 120, which may be used to predict the likelihood of readmission for a patient. The care transition model 120 may be trained using the historical discharge data and historical readmission data associated with the plurality of patients received from one or more of the healthcare facility systems 104a-104n. The care services system 102 may further include a prioritization element 122, which may determine a priority for the patient based on the likelihood of readmission and/or other criteria such as an expected cost of a readmission or a likelihood to contact the patient. The care services system 102 may further store and/or use various types of data used in performing the functions of the care services system 102. For example, the care services system 102 may store and/or access discharge data 126, interface data 128, and/or insurance data 130, in various examples.

In various implementations, the care services system 102 may include or use one or more hosts or combinations of compute resources, which may be located, for example, at one or more servers, cloud computing platforms, computing clusters, and the like. Generally, the care services system 102 is implemented by compute resources including hardware for memory 118 and one or more processors 116. For example, the care services system 102 may use or include one or more processors 116, such as a CPU, GPU, and/or programmable or configurable logic. In some embodiments, various components of the care services system 102 may be distributed across various computing resources, such that the components of the care services system 102 communicate with one another through the network 110 or using other communications protocols. For example, in some embodiments, the care services system 102 may be implemented as a serverless service, where computing resources for various components of the care services system 102 may be located across various computing environments (e.g., cloud platforms) and may be reallocated dynamically and/or automatically according to resource usage of the care services system 102, for example. In various implementations, the care services system 102 may be implemented using organizational processing constructs such as functions implemented by worker elements allocated with compute resources, containers, virtual machines, and the like.

The memory 118 may include instructions for various functions of the care services system 102 which, when executed by processor 116, perform various functions of the care services system 102. The memory 118 may further store data and/or instructions for retrieving data used by the care services system 102. Similar to the processor 116, memory resources used by the care services system 102 may be distributed across various physical computing devices. In some examples, memory 118 may access instructions and/or data from other devices or locations, and such instructions and/or data may be read into memory 118 to implement the care services system 102.

In various embodiments, memory 118 may store ADT data 126. ADT data 126 may include, in various examples, information relating to the admission of one or more patients to healthcare facilities, discharge of one or more patients from healthcare facilities after a health event, the transfer of one or more patients between healthcare facilities, and/or other inpatient events associated with one or more patients. Inpatient events may be identified by both insurance data 130 and ADT data 126. In some embodiments, inpatient events may include overlapping events (e.g. one claim/health information exchange (HIE) event starts before another claim/HIE event has ended) and exclude skilled nursing facility (SNF) records because SNF events almost always follow an inpatient hospitalization and may not represent a readmission to a healthcare facility. ADT data 126 may be stored at the memory 118 using various data structures. In some examples, ADT data 126 may further include instructions for accessing additional ADT data stored at another location accessible by the care services system 102, such as the datastore 108 of FIG. 1. Generally, ADT data 126 may include various types of information related to the discharge of a patient, such as reason for admission, medical condition, treatment, patient contact information, and/or other information related to a discharge of a patient from a healthcare facility.

In various embodiments, memory 118 may store interface data 128. Interface data 128 may include various types of information used by the care services system 102 to generate a healthcare facility system interface and/or an insurance system interface. For example, interface data 128 may include pre-structured queries to new ADT data from various healthcare facility systems 104a-104n and/or new insurance data from various insurance systems 106a-106n in communication with the care services system 102. In various examples, interface data 128 may further include one or more templates for generating a care transition display at a user interface 132 of a user device 114. For example, a template may include types of data to obtain from the healthcare facility systems 104a-104n, the insurance system 106a-106n, or from other locations to configure, for example, a care services interface.

In various embodiments, memory 118 may store insurance data 130. Insurance data 130 may be stored at the memory 118 using various data structures. In some examples, insurance data 130 may further include instructions for accessing additional insurance data stored at another location accessible by the care services system 102, such as the datastore 108 of FIG. 1. Generally, insurance data 130 may include various types of information related to the insurance associated with a patient, such as coverage, claims, claim status, and/or other information related to a patient's insurance.

The memory 118 may include instructions to implement a care transition model 120 when executed by the processor 116. The care transition model 120 may be a machine learning model, such as a neural network, a random forest classifier, gradient boosting model, linear regression, or another algorithm configured to predict the likelihood of readmission to a healthcare facility. For example, the care transition model 120 may calculate a likelihood of readmission within the next thirty days as a continuous value from 0 to 1 where 0 represents no likelihood of readmission in the next thirty days. The care transition model 120 may base the likelihood of readmission of a patient on the patient's discharge data. For example, the care transition model 120 may make a readmission prediction for a patient based on the patient's number of admissions to a healthcare facility in the last sixty to ninety days, the patient's number of readmissions to a healthcare facility within the last thirty days, and/or the patient's overall health.

In various examples, the care transition model 120 may be trained using historical discharge data and historical readmission data associated with a plurality of patients received from various healthcare facility systems 104a-104n. The care transition model 120 may also be trained using insurance data received from various insurance systems 106a-106n. For example, a health insurance claim associated with a patient may provide admission or readmission data associated with the patient when discharge and readmission data associated with the patient is incomplete. In various examples, the care transition model 120 may be further trained with additional readmission data associated with a patient obtained through the tracking of the patient, for example, by the care team. In various examples, the care transition model 120 may flag certain planned admissions to healthcare facilities, such as those associated with pregnancy, in the readmission profile.

The memory 118 may include instructions to implement a prioritization element 122 when executed by the processor 116. The prioritization element 122 may determine a priority for a patient based on the patient's likelihood of readmission. For example, a discharged patient with a high predicted likelihood of readmission may be assigned a higher priority than a discharged patient with a low predicted likelihood of readmission. In some examples, the patient's likelihood of readmission may be predicted by the care transition model 120.

In various examples, the prioritization element 122 may place patients in a workflow, for example, on a care services call list, in order of priority. In some embodiments, the prioritization element 122 may further base the priority for a patient on discharge data associated with the patient. For example, a discharged patient who was seen for a severe and/or ongoing health issue, such as a stroke, may be determined to have a higher priority than a discharged patient seen for a minor and/or temporary health issue, such as stitches on a finger.

In various examples, the prioritization element 122 may further base the priority for a patient on a likely cost of readmission associated with the patient. For example, a patient with a lower likely cost of readmission, such as a patient with a minor issue that is easily diagnosed and treated, may be determined to have a different priority than a patient associated with a higher cost of readmission. The prioritization element 122 may further base the priority for the patient on how likely it is that an intervention by a care team will prevent a readmission of the patient. For example, a patient discharged after a behavioral health event may have a lower chance of readmission for the behavioral health event if a care team member makes contact with the patient to follow up after the discharge. Whereas, a patient discharged after a health event resulting in a cancer diagnosis may have a high likelihood of readmission for cancer-related health events regardless of whether a care team member makes contact with the patient after the discharge. The prioritization element 122 may further base the priority for the patient on the likelihood that a care team member will be able to contact the patient.

The prioritization element 122 may create a prioritized workflow from the determined priorities and the care services system 102 may communicate the prioritized workflow to the user device 114 via the user interface 132. For example, the prioritization element may create a contact list of recently discharged and/or admitted patients in order of priority for a care team to use to follow up with the patients. In some examples, the prioritization element may generate a priority indicator based on the priority for the patient. The priority indicator may, for example, be a data tag, notification, or the like that is integrated into the readmission profile associated with the patient that is then used by the user, such as a care team, to follow up with the patient.

The memory 118 may include instructions implementing a systems interface 124 when executed by the processor 116. The systems interface 124 may generally communicate with systems external to the care services system 102 to receive data, access data, send data, and the like. For example, the systems interface 124 may communicate with the healthcare facility systems 104a-104n and/or the insurance systems 106a-106n to obtain additional discharge and/or insurance data. The systems interface 124 may communicate with other components of the care services system 102. For example, the systems interface 124 may communicate a new priority determination from the prioritization element 122 to the user device 114. Similarly, the systems interface 124 may communicate with the care transition model 120 to provide new discharge data received from various healthcare facility systems 104a-104n.

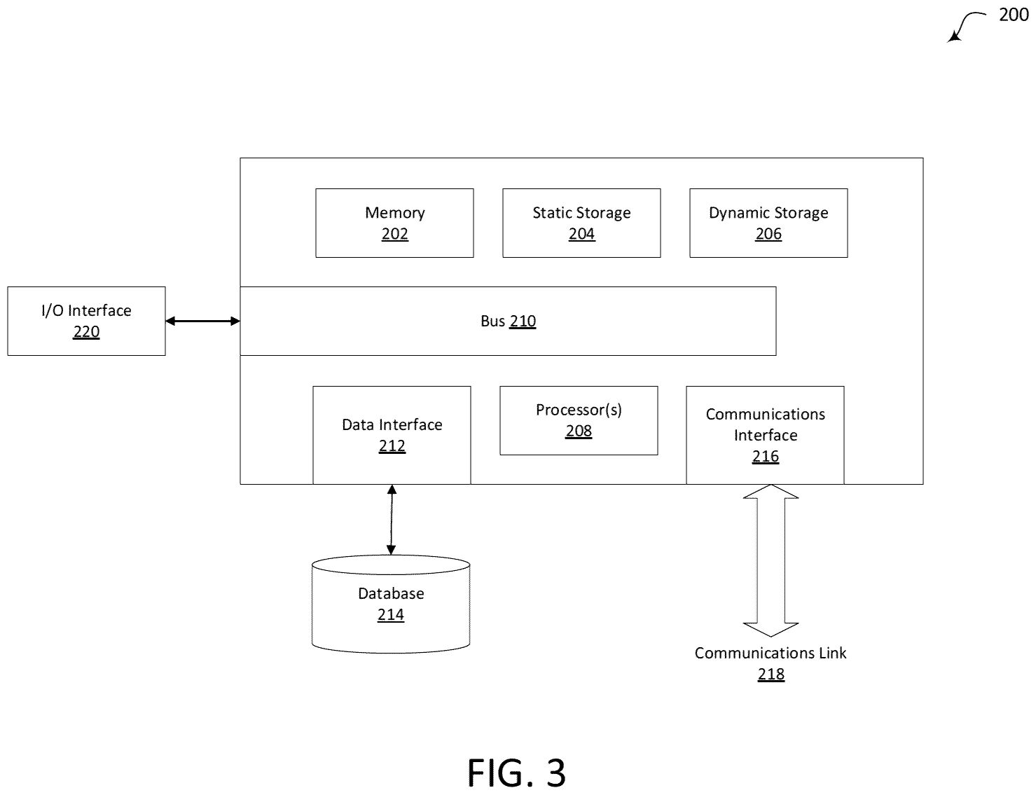

The care services system 102 may be implemented using various computing systems. Turning to FIG. 3, an example computing system 200 may be used for implementing various embodiments in the examples described herein. For example, processor 116 and memory 118 may be located at one or several computing systems 200. In various embodiments, the user devices 114 are also implemented by a computing system 200. Various systems, such as healthcare facility systems 104 and/or insurance systems 106 may be implemented by one or more computing systems 200. This disclosure contemplates any suitable number of computing systems 200. For example, the computing system 200 may be a server, a desktop computing system, a mainframe, a mesh of computing systems, a laptop or notebook computing system, a tablet computing system, an embedded computer system, a system-on-chip, a single-board computing system, or a combination of two or more of these. Where appropriate, the computing system 200 may include one or more computing systems; be unitary or distributed; span multiple locations; span multiple machines; span multiple data centers; or reside in a cloud, which may include one or more cloud components in one or more networks.

Computing system 200 includes a bus 210 (e.g., an address bus and a data bus) or other communication mechanism for communicating information, which interconnects subsystems and devices, such as processor 208, memory 202 (e.g., RAM), static storage 204 (e.g., ROM), dynamic storage 206 (e.g., magnetic or optical), communications interface 216 (e.g., modem, Ethernet card, a network interface controller (NIC) or network adapter for communicating with an Ethernet or other wire-based network, a wireless NIC (WNIC) or wireless adapter for communicating with a wireless network, such as a WI-FI network), input/output (I/O) interface 220 (e.g., keyboard, keypad, mouse, microphone). In particular embodiments, the computing system 200 may include one or more of any such components.

In particular embodiments, processor 208 includes hardware for executing instructions, such as those making up a computer program. The processor 208 circuitry includes circuitry for performing various processing functions, such as executing specific software for performing specific calculations and tasks. In particular embodiments, I/O interface 220 includes hardware, software, or both, providing one or more interfaces for communication between computing system 200 and one or more I/O devices. Computing system 200 may include one or more of these I/O devices, where appropriate. One or more of these I/O devices may enable communication between a person and computing system 200.

In particular embodiments, communications interface 216 includes hardware, software, or both providing one or more interfaces for communication (such as, for example, packet-based communication) between computing system 200 and one or more other computer systems or one or more networks. One or more memory buses (which may each include an address bus and a data bus) may couple processor 208 to memory 202. Bus 210 may include one or more memory buses, as described below. In particular embodiments, one or more memory management units (MMUs) reside between processor 208 and memory 202 and facilitate accesses to memory 202 requested by processor 208. In particular embodiments, bus 210 includes hardware, software, or both coupling components of computing system 200 to each other.

According to particular embodiments, computing system 200 performs specific operations by processor 208 executing one or more sequences of one or more instructions contained in memory 202. For example, instructions for the care transition model 120, prioritization element 122, and/or the systems interface 124 may be contained in memory 202 and may be executed by the processor 208. Such instructions may be read into memory 202 from another computer readable/usable medium, such as static storage 204 or dynamic storage 206. In alternative embodiments, hard-wired circuitry may be used in place of or in combination with software instructions. Thus, particular embodiments are not limited to any specific combination of hardware circuitry and/or software. In various embodiments, the term “logic” means any combination of software or hardware that is used to implement all or part of particular embodiments disclosed herein.

The term “computer readable medium” or “computer usable medium” as used herein refers to any medium that participates in providing instructions to processor 208 for execution. Such a medium may take many forms including but not limited to, nonvolatile media and volatile media. Non-volatile media includes, for example, optical or magnetic disks, such as static storage 204 or dynamic storage 206. Volatile media includes dynamic memory, such as memory 202.

Computing system 200 may transmit and receive messages, data, and instructions, including program, e.g., application code, through communications link 218 and communications interface 216. Received program code may be executed by processor 208 as it is received and/or stored in static storage 204 or dynamic storage 206 or other storage for later execution. A database 214 may be used to store data accessible by the computing system 200 by way of data interface 212. For example, ADT data 126, interface data 128, and/or insurance data 130 may each be stored using a database 214. In various examples, communications link 218 may communicate with, for example, user devices to display user interfaces to the care services system 102.

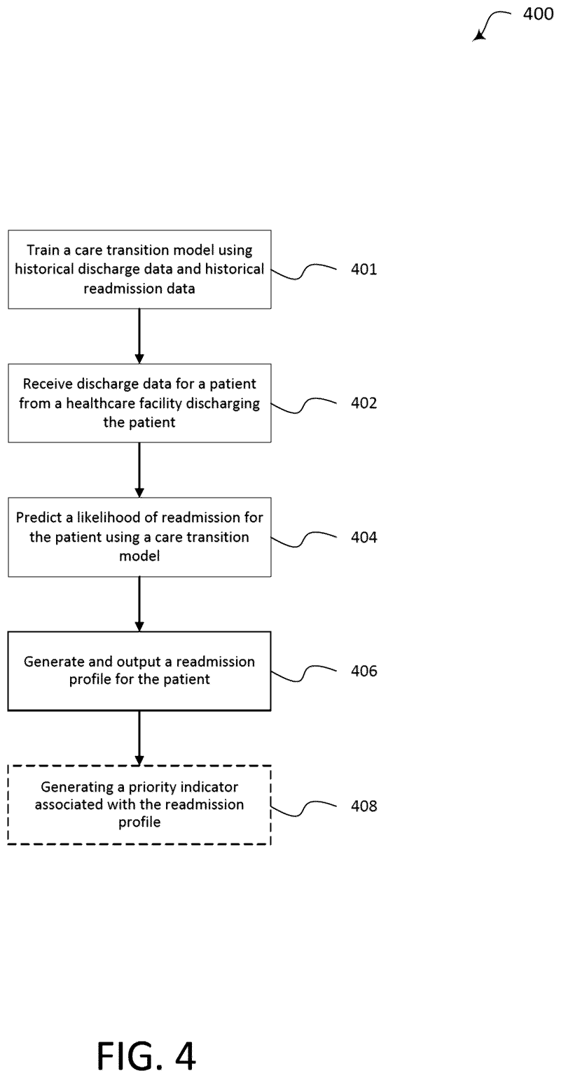

FIG. 4 illustrates an example process 400 for generation of a readmission profile for a discharged patient by a care services system, such as the care services system 102 of FIGS. 1 and/or 2. The process 400 may, in various examples, be used to generate the readmission profile in near-real time. That is, the readmission profile may be regularly updated and/or generated based on the most recent discharge data from a variety of healthcare facility systems (e.g., 104a-104n of FIG. 2) and/or insurance systems (e.g., 106a-106n of FIG. 2).

At block 401, the process 400 may include training a care transition model (e.g., 120 of FIG. 2) using historical discharge data and historical readmission data for a plurality of patients from a plurality of healthcare systems (e.g., 104a-104n of FIG. 2). The historical discharge data and the historical readmission data may be received by the care services system (e.g., 102 of FIGS. 1 and/or 2) by way of an ADT system, for example. In some embodiments, the care transition model may be further trained with data from a plurality of insurance systems (e.g., 106a-106n of FIG. 2). For example, the historical discharge data and the historical readmission data may be incomplete and the insurance data may backfill some of the missing historical data.

The historical discharge data and the historical readmission data may be used to train the care transition model to make predictions as to the likelihood of the readmission for a discharged patient. The data set may include information about patient demographics (e.g., age, gender, family status, housing status, and the like); information about previous admissions and discharges; information from insurance claims about the conditions members have; etc. The machine learning model is trained on the historical training data, for which it is known whether or not there was a readmission, to create a model which minimizes the error on the training set. Other characteristics that may be associated with discharge and readmission data may be included in the training set such as a lack of access to stable housing, food, and/or clean water.

In some embodiments a linear model may be used and the training set may be used to generate a set of weights for various characteristics or inputs. In these embodiments, the model may be configured to generate predictions by analyzing various input characteristics against the generated weighted attributes.

In some embodiments, the care transition model 120 may be further trained by inputting the historical discharge data associated with a patient in combination with historical readmission data associated with the patient that occurs within a time period after a discharge. For example, for a discharge of a patient from a healthcare facility for a health event, the number of readmissions of the patient to the same or to a different healthcare facility for the same health event within a time period after the discharge would be input into the care transition model 120 for training. The time period may be thirty days, sixty days, ninety days, and/or the like after the date associated with the discharge of the patient from the healthcare facility.

In some embodiments, the care transition model 120 may be further trained by inputting a patient's historical discharge data and historical readmission data in combination with the patient's health history, such as health events associated with each discharge and/or readmission. For example, a health event with a planned readmission, such as pregnancy, in the patient's health history may be treated differently than health events with unplanned readmissions, such as an infected wound.

In some examples, the care transition model 120 may be receive additional readmission data associated with a patient for additional training. The additional readmission data may be acquired through the tracking of the patient over a period of time, for example, by a care team.

At block 402, the process 400 may include receiving, by a processor (e.g., 116 of FIG. 2), discharge data for a patient from a healthcare facility system 104 discharging the patient after a health event. For example, the health event may be an illness, a traumatic injury, a mental health issue, a pregnancy, and the like. In various examples, the care services system 102 may be configured to receive such discharge data responsive to a discharge event on the healthcare facility system 104. In some examples, the care services system 102 may regularly query the healthcare facility system 104 to obtain new discharge data. The discharge data may, in some examples, be received via an application programming interface (API) associated with the healthcare facility. In some embodiments, the discharge data may be ADT data such as admission data or transfer data associated with the patient. For example, the care services system 102 may receive admission data for the patient from the healthcare facility system 104 admitting the patient.

At block 404, the process 400 may include predicting a likelihood of readmission for the patient using the care transition model 120. The care transition model 120, in some embodiments, may be a machine learning model such as a neural network. In various examples, the care services system 102 may predict the likelihood of readmission for the patient by inputting the current discharge data associated with the patient into the care transition model 120. In some embodiments, the care services system 102 may predict the likelihood of readmission for the patient following an upcoming discharge by inputting current admission data into the care transition model 120. In some examples, if available, other data associated with the patient may be input into the care transition model 120, such as health history, insurance, contact information, and/or the like.

At block 406, the process 400 may include generating and outputting a readmission profile for the patient to a user display (e.g., user devices 114 of FIGS. 1 and/or 2). In some embodiments, the readmission profile may be output to a user display via a user interface (e.g., 132 of FIG. 2). The readmission profile, for example, may include information such as the patient's likelihood of readmission, the patient's contact information, the patient's current discharge data, and the like. The likelihood of readmission may be based on readmission following a current discharge or based on readmission following an upcoming discharge calculated when the patient is admitted to the healthcare facility. In some embodiments, a care team member may use the readmission profile of a patient to create and execute a plan for following up with the patient. For example, if the readmission profile of the patient is created when the patient is admitted, the care team may follow up with the patient in person while they are in the healthcare facility.

In some embodiments, block 408 may be included in the process 400. At block 408, the process 400 may include generating a priority indicator associated with the readmission profile of the patient. The priority indicator, for example, may be a data tag, a notification, a data field, or the like. In some embodiments, the priority indicator may be based on the likelihood of readmission. For example, a patient with a low likelihood of readmission may be given a low priority. In some examples, the priority indicator for the patient may be further based on the likely cost of readmission associated with the patient. For example, a patient with a lower likely cost of readmission, such as a patient with a minor issue that is easily diagnosed and treated, may be given a different priority than a patient discharged for a major issue. In some examples, the priority indicator for the patient may be further based on other criteria such as the likelihood of readmission prevention and/or the likelihood that the care team will be able to contact the patient.

In some examples, the user may follow up with the patient while the patient is located at the healthcare facility based on the likelihood of readmission for the patient. For example, if the likelihood of readmission is generated based on the admission of the patient to the healthcare facility and indicates a high likelihood of readmission after the patient is discharged from the healthcare facility, the care team may make contact with the patient before the patient is discharged and while they are located in the healthcare facility.

In various examples, the priority indicator associated with the readmission profile of the patient may be used by a user, such a member of a care team, to place the patient on a priority list for follow up. In some examples, the care services system 102 may produce a call list of patients listed in order of priority for use by a user, such as member of a care team, to create and execute a plan for following up with the patient after a discharge from a healthcare facility.

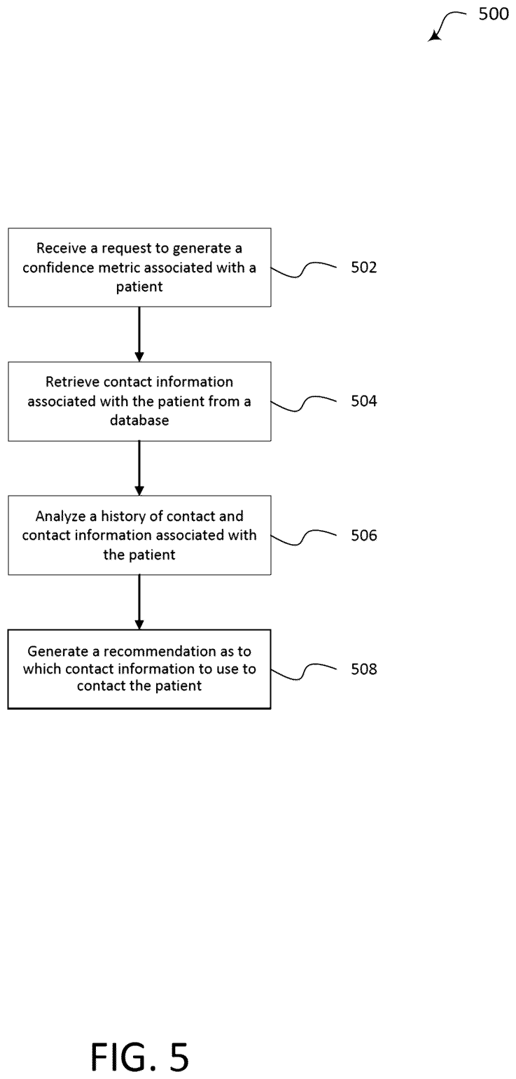

FIG. 5 illustrates an example process 500 for generation of a recommendation as to which contact information to use to contact a discharged patient by a care services system, such as the care services system 102 of FIGS. 1 and/or 2. The process 500 may, in various examples, be used to generate the contact information recommendation in near-real time. That is, the contact information may be regularly updated and/or generated based on the most recent ADT data from a variety of healthcare facility systems (e.g., 104a-104n of FIG. 2) and/or insurance systems (e.g., 106a-106n of FIG. 2).

At block 502, the process 500 may include receiving a request, by a processor such as 116 of FIG. 2, to generate a confidence metric associated with a patient discharged from a healthcare facility. The confidence metric may be a prediction as to the likelihood of reaching the patient. For example, the confidence metric may be the likelihood of a patient answering a telephone call from a care team. In some embodiments, the confidence metric may be predicted using a machine learning model. For example, the confidence metric may be integrated into the care transition model 120. In some embodiments, the request may come from a user of the care services system 102, such as a care team member. For example, the request may be generated using via a user interface (e.g., 132 of FIG. 2). It should be noted that although the term “metric” is used, the confidence assessment may be in other forms than a metric, e.g., a scoring, percentage, or likelihood of reaching a particular person with a particular contact method, such as a phone number or email address.

At block 504, the process 500 may include retrieving, by the processor (e.g., 116 of FIG. 2), contact information associated with the patient, such as from a database. The contact information associated with the patient may, in some examples, be stored by the care services system 102 (e.g., data 108 of FIG. 1). In some examples, the contact information may include contact information associated with discharge data and/or readmission data associated with the patient.

At block 506, the process 500 may include analyzing a history of contact and the contact information associated with the patient. For example, the history of contact associated with the patient may include information such as phone numbers used to contact the patient, which phone numbers successfully connected a caller with the patient, how often the patient has been successfully contacted and via what method, such as telephone, text message, email, letter, and the like. In some embodiments, the contact information may include several options for the patient, such as more than one address and/or more than one telephone number associated with the patient. Part of the analysis, for example, may include classifying the contact information, such as tagging the most recently gathered contact information and/or the contact information used with the most success. In various examples, the analysis may use factors such as the age of the patient, the language spoken by the patient, and/or other demographic features or information associated with the patient.

At block 508, the process 500 may include generating, by the processor (e.g., 116 of FIG. 2) a recommendation as to which contact information the user, such as a care team member, should use to follow up with the patient after a discharge from a healthcare facility. The recommendation, for example, may be based on the classification of the contact information. In some embodiments, the recommended contact information may be integrated into the readmission profile associated with the patient. In some embodiments, the recommended contact information may be used by the user, such as a care team member, to create and execute a plan for following up with the patient after a discharge from a healthcare facility. In some embodiments, the confidence metric may be generated when the patient is admitted to the healthcare facility and the recommendation may include contacting the patient while they are in the healthcare facility (e.g., before the patient is discharged). For example, the care team may contact the patient while they are in the healthcare facility to gather up-to-date contact information.

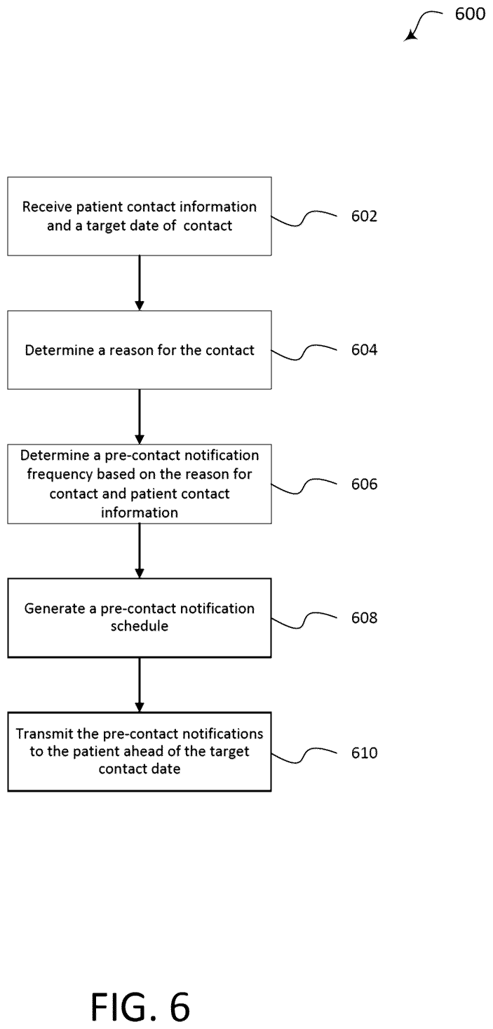

FIG. 6 illustrates an example process 600 for transmitting pre-contact notifications to a patient ahead of a target contact date. The target contact date, for example, may be based on the patient appearing on a call list to be contacted by a care team member for any particular reason, e.g., the target contact date may be based on a care team member's need to contact the patient in order to activate the patient's account with the care services system. In some embodiments, the target contact date may be a date determined for follow up by a care team member after the patient is discharged from a healthcare facility or regarding an ongoing health issue of the patient. In some examples, the target contact date may be based on a scheduled appointment for the patient. That is, the target contact date can be based on future “scheduled” health events or outreach or may be based on a real time new health event for which further contact or follow-up is needed.

In some embodiments, the process 600 may be used by users, such as care team members, to notify, in advance, patients with a scheduled follow up so that the patient may be prepared to engage with the care team member at the scheduled time. In some embodiments, the process 600 may be used to notify, in advance, patients without a scheduled appointment but that need to be contacted for another reason, such as activating the patient in the care services system, so that they may be prepared to engage with the care team member at a later time. In some embodiments, the pre-contact notification may be a general pre-contact notification, such as a welcome message after the patient is registered with the care services system, or a specific pre-contact notification, such as a message regarding a scheduled appointment.

At block 602, the process 600 may include receiving, by a processor (e.g., 116 of FIG. 2), contact information associated with a patient and receiving a target date of a contact associated with the patient. The target contact date may be a date on which the user of the care services system 102, such as a care team member, plans to interact with the patient. For example, the target contact date may be based on a scheduled interaction such as a scheduled appointment with the patient. In some embodiments, the target contact date may be based on a non-scheduled interaction such as the patient's name appearing on a call list to set up an account with the care services system.

At block 604, the process 600 may include determining, by a processor (e.g., 116 of FIG. 2), a reason for the contact of the patient. The reason, for example, may be to follow up with the patient regarding their discharge from a healthcare facility and/or the health event associated with the discharge. In some examples, the reason for the contact may be to update the patient's contact, demographic, and the like information. In some examples, the reason for the contact may be to follow up regarding an ongoing health event, such as a pregnancy, diabetes, or rehabilitation.

At block 606, the process 600 may include determining, by a processor (e.g., 116 of FIG. 2), a pre-contact notification frequency for the patient. In some embodiments, the pre-contact notification frequency may be based on the reason for the contact and/or the contact information associated with the patient. For example, some reasons for the contact may be determined to require fewer pre-contact notifications. In some embodiments, the pre-contact notification frequency may be a number of notifications in a day, a number of days that will include a pre-contact notification transmission, and/or another combination of time frames and numbers of pre-contact notifications.

In some embodiments, the pre-contact notification frequency may take into account general pre-contact notifications previously transmitted to the patient. For example, after registering with the care services system, the patient may receive a welcome letter as a general pre-contact notification. The welcome letter may be in the form of a physical posted letter, an email, a text message, and/or the like. The welcome letter may be included in the pre-notification frequency as one of the required notifications. In some embodiments, the welcome letter may be integrated with a specific pre-contact notification.

At block 608, the process 600 may include generating, by a processor (e.g., 116 of FIG. 2), a pre-contact notification schedule. In some embodiments, the pre-contact notification schedule may be generated by counting back a number of days before the target date of the contact and populating the number of days with the pre-contact notification transmissions based on the determined pre-contact notification frequency. For example, the pre-contact notification schedule for a patient may be one pre-contact notification transmission a day for the four days leading up to the target date of the contact. In some embodiments, the pre-contact notification schedule may only be created once a general pre-contact notification, such as a welcome letter, has been transmitted. In some embodiments, the target date of the contact is based on the patient appearing on a call list. For example, if a care team plans to contact a particular person, a pre-contact notification schedule may be created for the patient based on the patient appearing on a planned call list.

In some embodiments, the process 600 may include generating an indicator that a general pre-contact notification, such as a welcome letter, has been transmitted to a patient. For example, a call list may be generated listing patients that have had a general pre-contact notification transmitted to them. In some embodiments, the generated call list may be used by care team members to determine which patients to contact on a particular day. In some embodiments, the indicator may be integrated into an existing call list generated by the care services system listing patients that the team plans on calling on a particular day. In some embodiments, the indicator may be integrated into the readmission profile associated with the patient.

At block 610, the process 600 may include transmitting the pre-contact notifications to the patient ahead of the date of the scheduled contact according to the pre-contact notification schedule. In some embodiments, the pre-contact notifications may be transmitted as text messages, phone calls, pre-recorded voice messages, emails, and the like. In some examples, the type of pre-contact notification may be based on the contact information associated with the patient. For example, if the contact information associated with the patient only contains a phone number, the pre-contact notification may only be sent telephonically (e.g., a pre-recorded message, a text message (SMS), and the like) rather than via letter or another form. In some embodiments, the pre-contact notifications may be transmitted automatically, such as by a care services system 102. In some embodiments, the pre-contact notifications may be transmitted by a user, such as care team member using, for example, a call list generated by the care services system. In some embodiments, the pre-contact notifications may be transmitted from the same contact information as will be used to conduct the contact. For example, the patient may receive a welcome text from a particular phone number and that same phone number will be used to make the follow up contact.

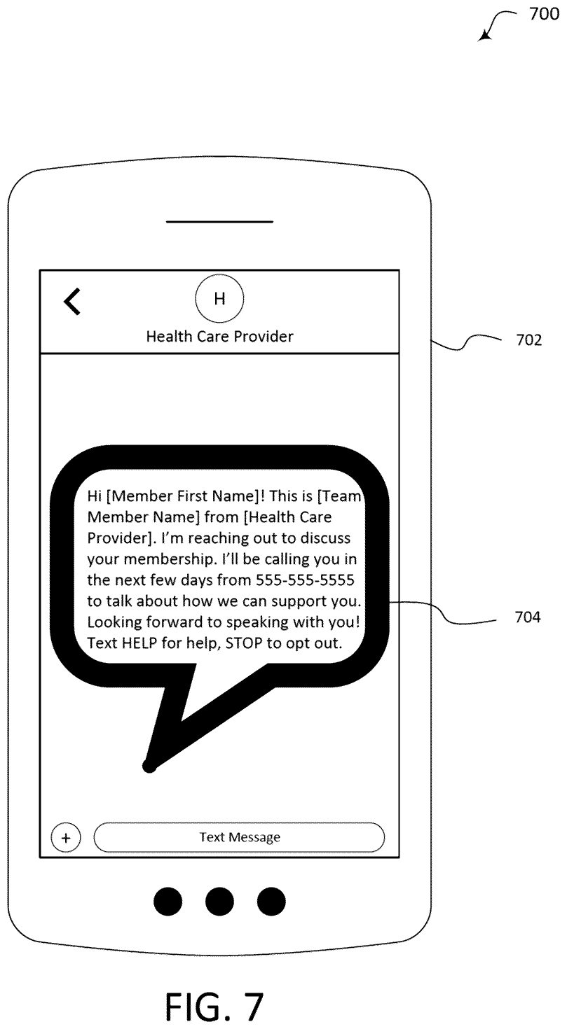

FIG. 7 depicts an example pre-contact notification according to some embodiments of the disclosure. In some embodiments, the pre-contact notification 704 may be transmitted in the form of a text message to be received on a device, such as phone 702. In other embodiments, the pre-contact notification 704 may be transmitted in other forms, such as by email, post, phone call, and the like. In some embodiments, the pre-contact notification 704 may be a general pre-contact notification, such as a welcome letter as is depicted by FIG. 7. In some embodiments, the pre-contact notification 704 may be a specific pre-contact notification that contains information pertaining to a specific subject associated with the patient, such as a follow up regarding a discharge from a healthcare facility after a health event.

Though the care services system 102 is described with respect to healthcare facilities and systems, the care services system 102 may be used by other similar businesses providing services to clients that require follow up by the business. For example, veterinary care providers.

The technology described herein may be implemented as logical operations and/or modules in one or more systems. The logical operations may be implemented as a sequence of processor-implemented steps directed by software programs executing in one or more computer systems and as interconnected machine or circuit modules within one or more computer systems, or as a combination of both. Likewise, the descriptions of various component modules may be provided in terms of operations executed or effected by the modules. The resulting implementation is a matter of choice, dependent on the performance requirements of the underlying system implementing the described technology. Accordingly, the logical operations making up the embodiments of the technology described herein are referred to variously as operations, steps, objects, or modules. Furthermore, it should be understood that logical operations may be performed in any order, unless explicitly claimed otherwise or a specific order is inherently necessitated by the claim language.

In some implementations, articles of manufacture are provided as computer program products that cause the instantiation of operations on a computer system to implement the procedural operations. One implementation of a computer program product provides a non-transitory computer program storage medium readable by a computer system and encoding a computer program. It should further be understood that the described technology may be employed in special purpose devices independent of a personal computer.

The above specification, examples and data provide a complete description of the structure and use of exemplary embodiments of the invention as defined in the claims. Although various embodiments of the claimed invention have been described above with a certain degree of particularity, or with reference to one or more individual embodiments, it is appreciated that numerous alterations to the disclosed embodiments without departing from the spirit or scope of the claimed invention may be possible. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

1. A computer implemented method comprising:
training a care transition model using historical discharge data and historical readmission data associated with a plurality of patients and a plurality of healthcare facilities;
receiving by a processor admission-discharge-transfer (ADT) data for a patient from a healthcare facility discharging the patient after a health event;
predicting by the processor a likelihood of readmission for the patient to a healthcare facility based on the ADT data using the care transition model; and
outputting a readmission profile for the patient to a user display, wherein the readmission profile for the patient is based on the likelihood of readmission for the patient.

2. The method of claim 1, wherein the likelihood of readmission is further based on a health history of the patient.

3. The method of claim 1, wherein the care transition model is further trained by:
tracking the patient over a period of time to obtain readmission data; and
providing the ADT data and the readmission data to the care transition.

4. The method of claim 1, further comprising determining by the processor a priority for the patient based on the likelihood of readmission.

5. The method of claim 4, wherein the priority is further based on a likely cost of readmission.

6. The method of claim 5, further comprising placing the patient on a priority list for follow up based on the priority.

7. The method of claim 1, wherein a confidence metric is generated by the care transition model.

8. The method of claim 1, wherein the ADT data is received from an application programming interface (API) associated with the healthcare facility.

9. One or more non-transitory computer readable media encoded with instructions which, when executed by one or more processors of a care services system, cause the care services system to:
receive discharge data for a patient from a healthcare facility discharging the patient;
predict a likelihood of readmission for the patient based on the discharge data using a care transition model, wherein the care transition model is trained using historical discharge data and historical readmission data associated with a plurality of patients and a plurality of healthcare facilities; and
determine a priority for the patient based on the likelihood of readmission.

10. The computer readable media of claim 9, wherein the likelihood of readmission is further based on a health history of the patient.

11. The computer readable media of claim 9, wherein the instructions further cause the care transition system to:
track the patient over a period of time to obtain readmission data; and
provide the discharge data and the readmission data to the care transition model as additional training data.

12. The computer readable media of claim 9, wherein the instructions further cause the care services system to:
place the patient on a priority list for follow up based on the priority.

13. The computer readable media of claim 9, wherein the priority is further based on a likely cost of readmission.

14. The computer readable media of claim 9, wherein the discharge data is received from an application programming interface (API) associated with the healthcare facility.

15. The computer readable media of claim 9, wherein a confidence metric is generated by the care transition model.

16. A computer implemented method comprising:
receiving by a processor a request to generate a confidence metric associated with a patient discharged from a healthcare facility after a health event;
retrieving by the processor contact information associated with the patient from a database;
analyzing by the processor a history of contact associated with the patient and the contact information associated with the patient; and
generating a recommendation as to which contact information to use to contact the patient.

17. The method of claim 16 further comprising:
analyzing demographic information associated with the patient.

18. A computer implemented method comprising:
receiving by a processor contact information associated with a patient and a target date of contact;
determining by the processor a reason for the contact;
determining by the processor a pre-contact notification frequency based on the reason for the contact and the contact information associated with the patient;
generating by the processor a pre-contact notification schedule; and
transmitting the pre-contact notifications to the patient ahead of the target date of the contact according to the pre-contact notification schedule.

19. The method of claim 18, wherein the pre-contact notifications are transmitted automatically.

20. The method of claim 18 further comprising determining the type of pre-contact notification based on the contact information associated with the patient.
